(19) United States
(12) Patent Application Publication
(10) Pub. No.: US 2011/0289641 A1
(43) Pub. Date: Dec. 1, 2011

(54) Visor With Sunglasses

(76) Inventor: EDWARD PISARCZYK, HAMILTON, NJ (US)

(21) Appl. No.: 13/115,718

(22) Filed: May 25, 2011

Related U.S. Application Data
(60) Provisional application No. 61/348,283, filed on May 26, 2010.

(51) Int. Cl. A61F 9/00

(52) U.S. Cl. 2/13

(57) ABSTRACT

A pair of sunglasses having an adjustable visor. The visor cuts down glare and is adjustable to a plurality of fixed positions for maximum user comfort. A clip friction hinge that joins the visor to the sunglasses frame allows a user a range of motion and a large number of fixed positions for the visor, thus enabling the user to adjust the visor to nearly any desired angle and to shade their eyes from glare in nearly any situation or at any time of day.

### CLAIM OF PRIORITY

This application claims priority to U.S. Ser. No. 61/348,283 filed May 26, 2010, the contents of which are fully incorporated herein by reference.

### FIELD OF THE INVENTION

The invention relates to improved sunglasses.

### BACKGROUND OF THE INVENTION

The invention relates to an adjustable sun visor attached to a pair of sunglasses. Presently, even the darkest sunglasses are ineffective against glare. Users may wear a hat or visor in addition to sunglasses to mitigate glare. Hats can be difficult to adjust relative to a user's face, can be uncomfortable, can muss a person's hair and can shift around or be blown off in the wind. Cloth visors face the same limitations. The present invention eliminates the need for a hat or cloth visor to prevent glare. In addition, the present invention has the advantage of being one relatively small item. This makes it easier for a user to carry from place to place and prevents the loss of hats or visors.

One unique feature of the present invention is the ability of the user to adjust the downward angle of the visor to a specific position through use of a pivotable hinge. Thus, unlike a hat or cloth visor, the user can shield his eyes at virtually any desired angle, and can change the angle of shielding as the sun moves through the sky.

Presently, there are different types of sunglasses for use in different circumstances and settings. For example, some sunglass lenses provide extremely good protection against very bright light, but hamper visibility at all other times. More sophisticated lenses offer a gradual darkening or lightening of lenses based on the intensity of the surrounding light conditions, but may not reduce glare. Examples of prior art are below.

U.S. Design Pat. 300,329; 308,979; 317,771; 339,597; 357,032; and 400,554 describe various designs of glasses with visors, but none appear adjustable.

U.S. Pat. No. 2,544,221 discloses a retrofit visor for vision correcting glasses that provides visor on the top and sides of the user's glasses. The visor is attached to the glasses using clips that engage the frame at various points.

U.S. Pat. No. 5,438,378 discloses a visor that includes thin, flat, opaque member. The rear of the member is concave to conform to a wearer's forehead. There is means for mounting the visor on the top of a pair of eyeglasses so the member extends forwardly in front of the eyeglasses and rearwardly to the wearer's forehead. The means may be a pair of spaced-apart clips extending from the bottom of the member intermediate the front and the rear thereof. Each clip has a bottom, an aperture above the bottom and a slit extending from the aperture to the bottom of the clip. The slit is narrower than the aperture. The top of the frame of a pair of eyeglasses is receivable in the aperture of each clip to secure the visor to the eyeglasses.

U.S. Pat. No. 5,519,460 discloses a pair of sunglasses for shielding glare and filtering light directed into human eyes. The inventive device includes a frame having ear pieces pivotally mounted thereto. A lens is removably mounted to the frame for filtering light passing through the lens. A visor is mounted to the frame so as to project outwardly therefrom to reduce glare into the eyes of the wearer.

U.S. Pat. No. 5,524,291 discloses a sun visor for eye glasses, comprising a U-shape or arcuate frame member which supports a stretchable fabric covering, in which the fabric covering includes an upper and lower panel to provide a pocket therebetween. The upper and lower panels are sewn together around the arcuate or U-shape portion of their periphery in a pair of spaced apart peripheral seams that provides a peripheral through passageway for the U-shaped or arcuate frame member. The opposite end portions of the arcuate frame member project outwardly from each opposite opening to the peripheral through passageway. A strip of material having tiny hook members thereon is secured to the inwardly facing surface of each outwardly projecting end portion for releasably connecting to corresponding members having tiny loops thereon secured to the forward ends of the temple pieces of the eye glasses, to releasably secure the sun visor to the eye glasses. A relatively stiff but flexible supporting strip is secured to the laterally extending edge of the lower fabric panel, slightly longer than such edge whereby the strip has to be bowed slightly in the upward direction which in turn biases the laterally extending edge of the lower panel against the laterally extending edge of the upper panel, thereby biasing the entrance to the pocket towards the closed position. A retaining flap extends inwardly of the pocket from the laterally extending edge of the upper panel.

Various sunglasses, as well as vision correcting eyeglasses with visors, are known in the art. None of the art relating to visors pertains specifically to sunglasses, and the art also does not pertain to the present invention in which sunglasses are preferably designed to support a pivoting hinge. One embodiment of this invention is illustrated in the accompanying drawings and will be described in more detail herein below.

### SUMMARY OF THE INVENTION

The present invention discloses an apparatus with a visor, said visor having a hinge connected to a sunglasses frame, wherein said hinge is capable of pivoting to a plurality of fixed positions at various angles with respect to said sunglasses frame. The sunglasses and visor are one piece, making it easy to use, as the visor is designed to be joined at a frame portion of the sunglasses. The hinge is a unique feature that allows the visor to be placed in any desired vertical position to shield a user from the sun's glare.

Therefore, the present invention succeeds in conferring the following desirable and useful benefits and objectives.

It is an object of the present invention to provide a visor for sunglasses that is capable of eliminating glare.

It is another object of the present invention to provide a visor for sunglasses whose angle above the lenses is adjustable.

Yet another object of the present invention is to provide a visor capable of enhancing the vision protection supplied by sunglasses.

Still another object of the present invention is to provide a visor that obviates the need to wear a cap or hat that contains an integral visor.

Yet another object of the present invention is to provide an inexpensive device that is capable of enhancing the effectiveness of sunglasses without greatly affecting their cost.

Still another object of the present invention is to provide a device that is capable of functioning as an attractive yet functional accessory for sunglasses.

### BRIEF DESCRIPTION OF THE DRAWINGS

FIG. 1 is a perspective view of the present invention attached to a pair of sunglasses.

FIGS. 2A and 2B is a detailed cross sectional view of the pivot.

FIG. 3 is a top view of the present invention that is attached to a pair of sunglasses.

FIG. 4 is an exploded view of the present invention.

### DESCRIPTION OF THE PREFERRED EMBODIMENTS

Reference will now be made in detail to embodiments of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

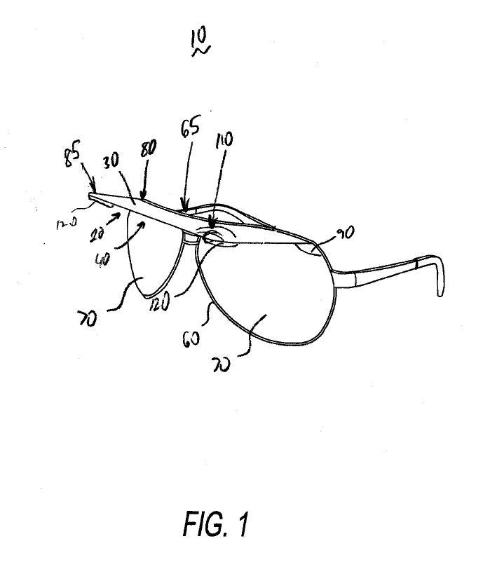

Referring to FIG. 1, shown is an apparatus 10 with a visor 20 that has a first surface 30, a second surface 40, a top portion 80, a bottom portion 85, an angle 90, and a cushion element 120. Also shown is a sunglasses frame 60, with frame top center portion 65 and lenses 70. The visor is one of many positions that shield the user's eyes from glare.

The visor is preferably opaque. However, it may also be made from a heavily tinted, but still transparent material. The preferred material for the visor 20 may be, but is not limited to plastic, polarized plastic, glass, resin, metal or wood, or any other polymeric material or composites of materials. Additionally, the visor 20 may be coated with a light reflecting material to direct the sun's rays away from the user's face.

The first surface 30 faces outward and is intended to block any direct light radiation that is emitted by the sun or by an artificial light source. The first surface 30 may be composed of a reflective surface or may be utilized to display a picture or a logo. The second surface 40 faces downward or toward the lenses 70. The second surface 40 should be substantially dark and preferably not reflective, to further eliminate any light rays reflected by the ground or by objects surrounding the wearer. The two surfaces 30 and 40 thus function to eliminate direct and reflected glare. It is preferable that both the first surface 30 and the second surface 40 are made from dark, dense plastic. These two surfaces may form separate layers of the visor 20 or may be two separate but attached components.

When fully raised, the visor 20 forms an angle relative to the sunglasses lenses that is slightly greater than 90 degrees. This angle can be increased by raising the visor 20 further toward the user's forehead, or decreased by lowering the visor 20 toward the user's chin. An angle adjustment is highly desirable to take into account times of the day when the sun is hovering just above the horizon. It may also be very useful to reduce sunlight reflected off of water, glass surfaces, ground level items and passing vehicles. The light deflecting component 20 may be closed entirely, such that the second surface 40 abuts the sunglasses frame 60 or the lenses 70. In such an orientation the light deflecting component 20 may also form a cover for the lenses 70. This allows for protection of the lenses 70 as well as providing a compact way to store and carry the apparatus.

FIG. 1 shows the holding surface 110 and the cushion element 120. Both components are optional. The holding surface 110 may provide an opaque or frictional surface that can be grasped when rotating the light deflecting component 20. This guarantees that an optimal angle 90 is set quickly, without fingerprint impressions marring the appearance of the first surface 30. The holding surface 110 is an optional but preferred component. Another optional component is the cushion element 120, which may protect the lenses 70 and the frame 60 from scratches and other damage when the light deflecting component 20 is completely folded over the frame 60. The light deflecting surface 20 should preferably span the width of the sunglasses frame 60 and be contoured to substantially conform to the shape of the lenses 70.

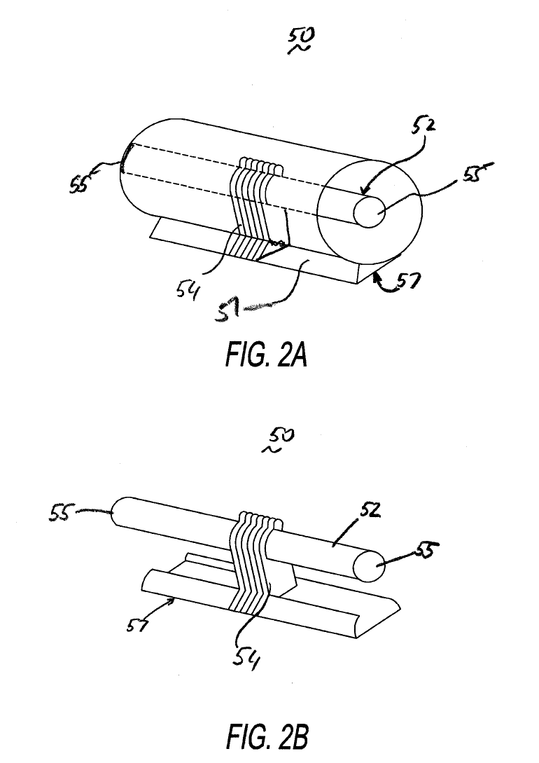

FIGS. 2A and 2B are preferred embodiments for the hinge 50. The hinge 50 shown in FIGS. 2A and 2B is a clip friction hinge and is described in greater detail in U.S. Pat. No. 5,697,125, which is fully incorporated herein by reference. The hinge 50 enables the visor 20 to swing up or down to attain a desired angle 90; the design of the preferred embodiment shown in FIGS. 2A and 2B holds the visor at a desired position, so once the desired angle is attained the visor stays stationary.

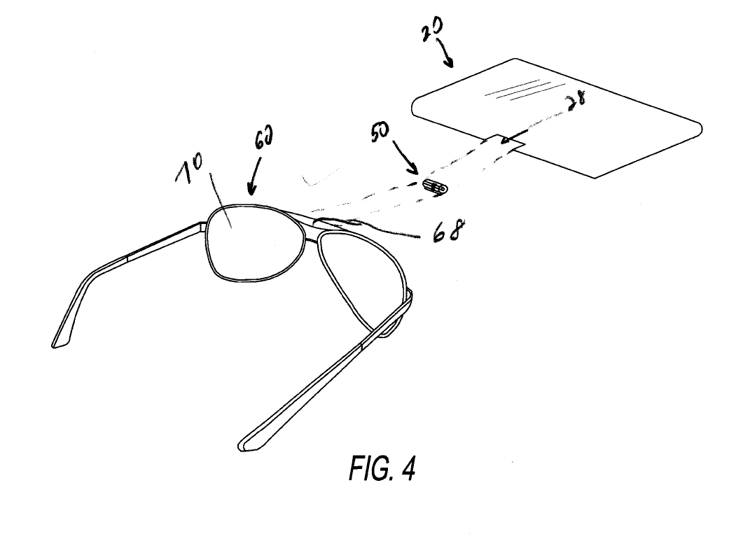

The hinge in FIGS. 2A and 2B contains a shaft 52 with shaft ends 55, holding elements 54, and a base surface 57. The holding elements 54 are biased towards closure and therefore maintain a tight grip on the shaft 52. The base surface 57 may be connected to the frame 60 using a variety of devices, including but not limited to, fasteners, rivets, welding, crimping, soldering or adhesives. The base surface 57 may also contain a pin, a clamp or magnets to detachably connect to the frame 60. The ends of the shaft 55 may be inserted into a slot 22 (FIG. 4) or attached to arms (not shown) that may be extending from the visor 20. Although the preferred hinge is the clip friction hinge, other hinges may be used and may include, but are not limited to, friction hinges, such as but not limited to, a spring hinge, and an axial compression hinge. Other devices for achieving the desired result may also be used, including but not limited to, a ball and socket or other type of hinge.

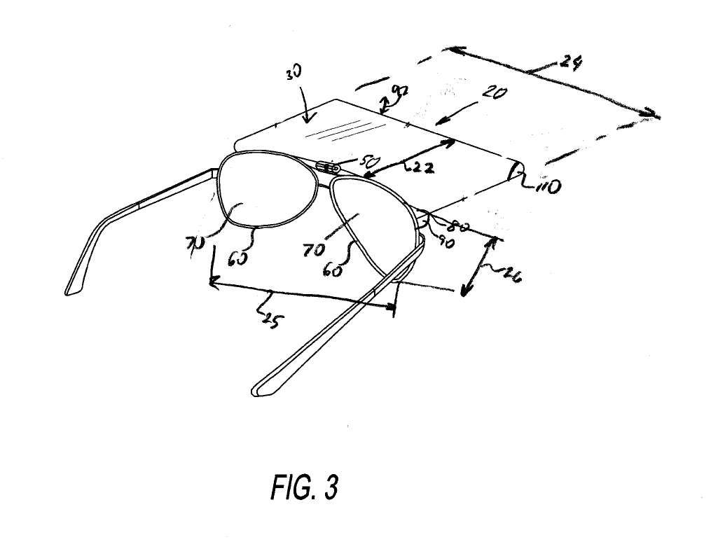

FIG. 3 is a top view of the present invention. Shown is apparatus 10, with visor 20, which has a visor width 22, a visor length 24, a first surface 30, a top portion 80, and a holding surface 110. Also shown is sunglasses frame 60, with lenses 70. The sunglasses frame 60 has a frame width 25 and a frame length 26; the lenses have a lens height 26. Hinge 50 and angle 90 are also depicted in FIG. 3.

The preferred visor width 22 may be between ¾″ and 10 inches, the preferred visor length 24 may be between 3 and 12 inches, and the preferred visor thickness is between 1/16″ and ¾″ inches. Alternatively, the visor length 24 may be slightly narrower or slightly wider than the frame width 25, while the visor width 22 may be slightly lesser then or slightly greater than the lens height 26.

The top portion 80 of the visor 20 is attached to a hinge 50, which in turn is attached to the frame 60. The attachments may be with any device that achieves the purpose, including but not limited to, fasteners, welding or adhesives. The hinge is preferably attached at the frame top center portion 65. However, the hinge may be disposed anywhere on the frame, and there may be additional hinges oriented along the top portion of the frame 60.

FIG. 4 is an exploded view of the apparatus 10. FIG. 4 shows the visor 20, visor slot 28, hinge 50, sunglasses frame 60, frame attachment bar 68, and sunglasses lenses 70. The visor 20 is attached to the hinge 50 which is then attached to the frame attachment bar 68. As noted above, any method may be used for these attachments. Sunglasses may be manufactured specifically to be used with the visor, in which case the hinge may be permanently attached to the visor, the sunglasses frame, or both.

Alternately, the visor may be retroactively fitted to a pair of sunglasses. It still could be permanently attached, or it may be removably attached using a clip or other method. If a pair of sunglasses does not contain a piece that could be used as a frame attachment bar, then the frame attachment bar could be added to the sunglasses frame with the visor and hinge. Although shown in a specific position on the sunglasses frame, the frame attachment bar could be anywhere on the sunglasses frame. Additionally, the portion of frame to which the hinge and visor are attached need not be a bar but may be any shape or any type of attachment that accommodates the hinge.

The various parts of the present invention may be made from any material, including but not limited to: plastics and resins including but not limited to plastic, rubber, foam, silicone, ABS, Polycarbonate, Noryl™, PVC, Polystryrene, ABS/PVC, PVC/Acrylic, Polysulfone, Acrylic, Polyethylene, Kydex™, PETG; glass, including but not limited to fiberglass, borosilicate, or quartz; wood; metals, including but not limited to iron, tin, aluminum, copper; rubber including but not limited to natural rubber, SBR, Isoprene rubber, Butadiene rubber, and Chloroprene rubber; cloth, leather or textiles; or any combinations or composites of these materials or other materials and new materials that may be manufactured in the future. The parts of the apparatus 10 may be manufactured using identical or different materials.

The visor 20 is shown as a rectangular shape, but it may be any shape, including but not limited to, square, oval, oblong, circle or multiple circles, star or multiple stars, animal shape, or any shape a user desires. The visor and hinge 20 may be any color or combination of colors or designs. The visor may contain indicia, pictures, or drawings, either on one side or one both sides.

The present invention may be used with existing embodiments of glasses or sunglasses, or may be integral with a pair of sunglasses. The disclosed apparatus 10 may be temporarily or permanently attached to an existing frame 60. When sunlight is very bright, the visor 20 may be lowered. When conditions become more overcast or darker, the visor 20 may be raised. When the sunglasses are not in use, the visor 20 may be completely folded down to function as a protective shield for the lenses 70. The visor 20 may be manufactured from materials that are light absorbing, light deflecting, or neutral.

Although this invention has been described with a certain degree of particularity, it is to be understood that the present disclosure has been made only by way of illustration and that numerous changes in the details of construction and arrangement of parts may be resorted to without departing from the spirit and the scope of the invention.

What is claimed:

1. An apparatus comprising:
a substantially opaque visor, said visor having a clip friction hinge connected to a center portion of a frame attachment bar of a sunglasses frame, wherein said hinge is capable of pivoting to, and remaining substantially fixed at, a plurality of positions such that a plane of said visor is oriented at various angles with respect to said sunglasses frame, and wherein said various angles are in a range of substantially zero degrees to ninety degrees.

2. The apparatus of claim 1, wherein said visor is made from polarized plastic.

3. The apparatus of claim 1, wherein said visor further comprises a holding surface.

4. The apparatus of claim 1, wherein said second surface further comprises a cushion element, said cushion element being disposed on an outer edge of said second surface.

5. The apparatus of claim 1, wherein said visor forms a protective cover for lenses.

6. The apparatus of claim 1, wherein said visor attaches to said pivot in a detachable fashion.

7. The apparatus of claim 1, wherein said pivot is attached to said sunglasses frame in a detachable fashion.
